(19) United States
(12) Patent Application Publication
(10) Pub. No.: US 2023/0088941 A1
(43) Pub. Date: Mar. 23, 2023

(54) INFORMATION PROCESSING APPARATUS, INFORMATION PROCESSING METHOD, AND NON-TRANSITORY COMPUTER READBLE STORAGE MEDIUM

(71) Applicant: MEDIASEEK, inc., Tokyo (JP)

(72) Inventors: Naoki NISHIO, Tokyo (JP); Ryoki KOGO, Tokyo (JP); Yuki HIRAI, Tokyo (JP); Tetsuro SAITO, Tokyo (JP)

(21) Appl. No.: 17/822,432

(22) Filed: Aug. 26, 2022

(30) Foreign Application Priority Data
Sep. 21, 2021 (JP) 2021-152840

(51) Int. Cl. A61M 21/02; A61B 5/00

(52) U.S. Cl. CPC A61M 21/02; A61B 5/0022; A61M 2021/0016

(57) ABSTRACT

An information processing apparatus includes a storage that stores analysis method information indicating an analysis method of brain waves of a user and notification method information indicating a method for notifying a state of brain waves in association with each of a plurality of user states indicating a symptom or ability of the user, a selection part that selects an improvement target state that is a user state that is to be improved by a target user, an acquisition part that acquires brain wave information of the target user, a state identification part that identifies a state of brain waves indicated by the brain wave information according to an analysis method associated with the selected improvement target state, and a notification part that notifies the target user of the state of the brain waves on the basis of the notification method associated with the selected improvement target state.

### CROSS-REFERENCE TO RELATED APPLICATIONS

The present application claims priority to Japanese Patent Applications number 2021-152840, filed on Sep. 21, 2021. The contents of this application are incorporated herein by reference in their entirety.

### BACKGROUND OF THE DISCLOSURE

The present disclosure relates to an information processing apparatus, an information processing method, and a program for performing processing related to brain wave information indicating brain waves.

Neurofeedback in which brain function is increased by making a person whose brain waves are measured recognize a measurement state of the brain waves and performing training to adjust the brain waves as intended by the person is known. For example, Japanese Unexamined Patent Application Publication (Translation of PCT Application) No. 2020-501625 discloses an apparatus for performing neurofeedback, which is training for causing the person to correct his brain activity so that brain waves when the person is relaxed are detected.

Neurofeedback includes not only training for enabling a person to generate brain waves that make the person relaxed, but also training for enabling generating brain waves to enhance concentration, and training for enabling generating brain waves to relieve dementia and depression, and analysis methods of brain waves are different for each of these types of training. However, an apparatus capable of analyzing brain waves according to an analysis method corresponding to an ability or symptom that a user wants to improve and capable of performing training for increasing brain functions has not been provided in the past.

### BRIEF SUMMARY OF THE DISCLOSURE

The present disclosure has been made in view of these points, and an object of the present disclosure is to enable performance of training for increasing brain functions according to the ability or symptom that the user wants to improve.

An information processing apparatus according to the first embodiment of the present disclosure include a storage that stores analysis method information indicating an analysis method corresponding to an analysis of brain waves of a user and notification method information indicating a notification method for notifying the user of a state of the brain waves of the user identified by the analysis, in association with each of a plurality of user states indicating a symptom or ability of the user, a selection part that selects an improvement target state that is a user state that is to be improved by a target user whose brain waves are detected, among the plurality of user states, an acquisition part that acquires brain wave information indicating brain waves of the target user, a state identification part that identifies a state of the brain waves of the target user indicated by the brain wave information according to an analysis method indicated by the analysis method information stored in the storage in association with an improvement target state selected by the selection part, and a notification part that notifies the target user of the state of the brain waves identified by the state identification part on the basis of the notification method indicated by the notification method information stored in the storage in association with the improvement target state selected by the selection part.

An information processing method according to the second embodiment of the present disclosure, executed by a computer, includes the steps of selecting an improvement target state that is a user state that is to be improved by a target user whose brain waves are detected, from a plurality of user states indicating a symptom or ability of a user, acquiring brain wave information indicating brain waves of the target user, identifying a state of the brain waves of the target user indicated by the brain wave information according to an analysis method indicated by the analysis method information stored in association with the selected improvement target state, in a storage that stores analysis method information indicating an analysis method corresponding to an analysis of brain waves of the user and notification method information indicating a notification method for notifying the user of a state of brain waves of the user identified by the analysis, in association with each of a plurality of user states, and notifying the target user of the identified state of the brain waves on the basis of the notification method indicated by the notification method information stored in the storage in association with the selected improvement target state.

A storage medium according to the third embodiment of the present disclosure stores, in a non-transitory manner, a program for causing a computer to function as a selection part that selects an improvement target state that is a user state that is to be improved by a target user whose brain waves are detected, from a plurality of user states indicating a symptom or ability of a user, an acquisition part that acquires brain wave information indicating brain waves of the target user, a state identification part that identifies a state of the brain waves of the target user indicated by the brain wave information according to an analysis method indicated by the analysis method information stored in association with the selected improvement target state, in a storage that stores analysis method information indicating an analysis method corresponding to an analysis of the brain waves of the user and notification method information indicating a notification method for notifying the user of a state of the brain waves of the user identified by the analysis in association with each of the plurality of user states, and a notification part that notifies the target user of the identified state of the brain waves on the basis of the notification method indicated by the notification method information stored in the storage in association with the selected improvement target state.

### BRIEF DESCRIPTION OF THE DRAWINGS

FIG. 1 shows an outline of an information processing system according to the first embodiment.

FIG. 2 shows a configuration of the information processing apparatus according to the first embodiment.

FIG. 3 shows an example of combination information.

FIG. 4 is a flowchart showing a flow of processing in the information processing apparatus according to the first embodiment.

FIG. 5 shows a configuration of the information processing apparatus according to the second embodiment.

FIG. 6 shows a configuration of the information processing apparatus according to the third embodiment.

### DETAILED DESCRIPTION OF THE DISCLOSURE

Hereinafter, the present disclosure will be described through exemplary embodiments, but the following exemplary embodiments do not limit the disclosure according to the claims, and not all of the combinations of features described in the exemplary embodiments are necessarily essential to the solution means of the disclosure.

### First Embodiment

### [Outline of Information Processing System S]

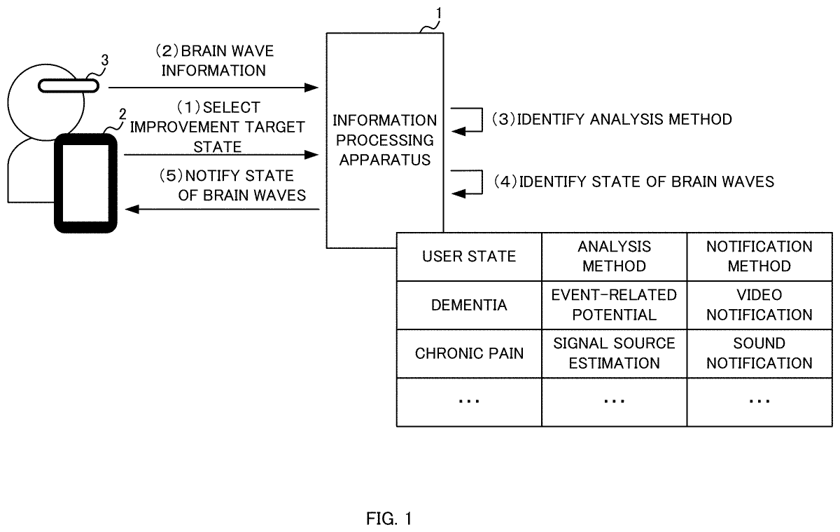

FIG. 1 shows an outline of an information processing system S according to the first embodiment. The information processing system S is a system for performing training for increasing brain functions of a user. As shown in FIG. 1, the information processing system S includes an information processing apparatus 1, a user terminal 2, and a brain wave measuring device 3. The information processing apparatus 1 is connected to the user terminal 2 possessed by a target user whose brain waves are measured, in a manner enabling communication via a communication network, such as an Internet line or a mobile phone network. The information processing apparatus 1 is a computer that identifies a state of brain waves of the target user and notifies the user terminal 2 of the state of the brain waves.

The user terminal 2 is a terminal such as a smartphone, a tablet, or a personal computer possessed by a user. The user terminal 2 is connected to the brain wave measuring device 3 that measures brain waves of the target user, in a manner enabling communication via short-range wireless communication, such as Bluetooth (registered trademark), wireless communication, such as wireless LAN, or wired communication, such as a USB (registered trademark).

In the first embodiment, the information processing apparatus 1 selects an improvement target state by receiving, from the user terminal 2, a selection of the improvement target state that is a user state that is to be improved by the target user whose brain waves are detected, among a plurality of user states indicating a symptom or ability of the user ((1) in FIG. 1). The improvement target state is a state where there is room for improvement, such as a state where short-term memory is decreased, a state of having dementia, a state of having chronic pain, or a state of having depression.

The information processing apparatus 1 acquires brain wave information indicating the brain waves of the target user measured in the brain wave measuring device 3 via the user terminal 2 ((2) in FIG. 1). The information processing apparatus 1 stores analysis method information indicating an analysis method corresponding to an analysis of brain waves and notification method information indicating a method for notifying the user of the state of the brain waves of the user, in association with each of the plurality of user states. The information processing apparatus 1 identifies an analysis method used for an analysis of brain waves by identifying analysis method information associated with the selected improvement target state ((3) in FIG. 1).

The information processing apparatus 1 identifies the state of the brain waves of the target user indicated by the brain wave information through the identified analysis method ((4) in FIG. 1). The information processing apparatus 1 identifies the notification method indicated by the notification method information associated with the selected improvement target state. The information processing apparatus 1 notifies the user terminal 2 of the identified state of the brain waves using the identified notification method, thereby notifying the target user of the identified state of the brain waves ((5) in FIG. 1).

In this manner, the information processing system S selects an analysis method and a notification method that are considered to be appropriate for an ability or symptom the user wants to improve, making it possible to effectively perform training for increasing brain functions corresponding to the ability or the symptom the user wants to improve.

[Configuration of information processing apparatus 1]

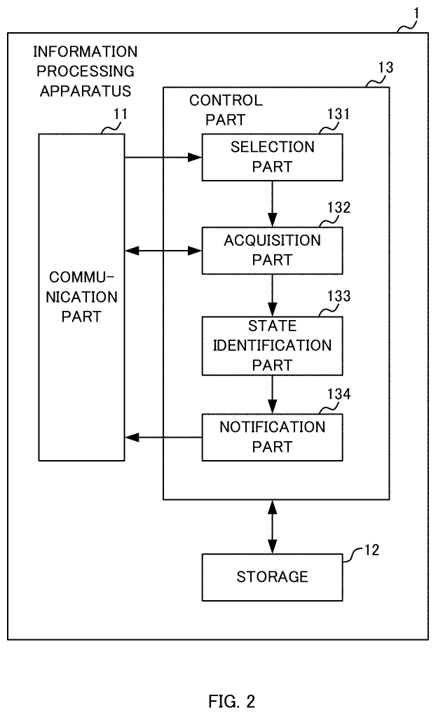

Next, a configuration of the information processing apparatus 1 will be described. FIG. 2 shows the configuration of the information processing apparatus 1 according to the first embodiment. As shown in FIG. 2, the information processing apparatus 1 includes a communication part 11, a storage 12, and a control part 13.

The communication part 11 is an interface for connecting to a communication network, such as an Internet line or a mobile phone network.

The storage 12 is a storage medium including a Read Only Memory (ROM), a Random Access Memory (RAM), or the like. The storage 12 stores a program executed by the control part 13. The storage 12 stores a program that causes the control part 13 to function as a selection part 131, an acquisition part 132, a state identification part 133, and a notification part 134, for example.

Further, the storage 12 stores combination information in which analysis method information, indicating an analysis method for analyzing brain waves of the user, and notification method information, indicating a method for notifying the user about the state of the brain waves of the user identified by the analysis, are associated with each of a plurality of user states indicating a symptom or ability of the user.

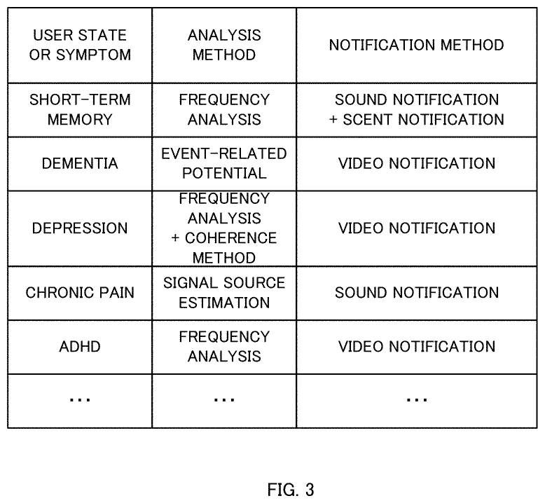

FIG. 3 shows an example of combination information. As shown in FIG. 3, it can be confirmed that the analysis method information and the notification method information are associated with each of the plurality of user states. In FIG. 3, names of notification methods for identifying the notification method are shown as the notification method information. The symptom or ability of the user is a symptom or ability that is improved by training brain functions. The symptom or ability of the user includes short-term memory, dementia, depression, chronic pain, and attention-deficit hyperactivity disorder (ADHD), as shown in FIG. 3. Further, the analysis method includes a frequency analysis, a signal source estimation, a coherence method, an event-related potential, and a combination of these analysis methods.

The frequency analysis is an analysis method for performing a frequency spectrum analysis of brain waves and analyzing the intensity of the brain waves for each frequency band, such as alpha waves, beta waves, and theta waves, for example. The signal source estimation is an analysis method for analyzing the source of brain waves on the basis of brain waves measured from a plurality of locations on the head of the target user by the brain wave measuring device 3. The brain function being activated can be identified by analyzing the source of the brain waves.

The coherence method is an analysis method in which the alignment degree of the phase of brain waves measured at two portions of the head is identified and a network formed in the brain is analyzed on the basis of the alignment degree. The event-related potential is an analysis method for identifying brain waves that occur in response to an act of viewing an object or thinking about something by the target user, for example.

An analysis of brain waves using the frequency analysis is considered to be suitable for improving short-term memory, which is one of the user states, for example. Accordingly, as shown in FIG. 3, it can be confirmed that the frequency analysis is associated with the short-term memory as the analysis method. An analysis method that is considered effective is also associated with each of the other user states.

The notification method information includes the names of the notification methods, and also includes information indicating a combination of a type of content used for notifying the state of the brain waves and a mode of changing components of the content on the basis of the state of the brain waves, as shown in FIG. 3. The type of the content is “sound”, “video”, and “scent”, and the mode of changing the content includes “change in quality” and “change in strength of scent”, for example.

Further, the notification method includes a sound notification as the first notification method, a video notification as the second notification method, a scent notification as the third notification method, and a notification method in which at least two notification methods from the first notification method to the third notification method are combined, as the fourth notification method.

The notification method information indicating the sound notification as the first notification method includes information indicating a combination of “sound” as a type of content used for notifying the state of the brain waves and “change in quality” as a mode of changing components of the content, for example. The sound notification outputs a sound, which is the content, to the user terminal 2 on the basis of the information indicating the combination, and notifies the target user of the state of the brain waves by adding a noise to the content on the basis of the state of the brain waves of the target user.

Further, the notification method information indicating the video notification as the second notification method includes information indicating a combination of “video” as a type of content used for notifying the state of the brain waves and “change in quality” as a mode of changing components of the content. The video notification outputs a video, which is the content, to the user terminal 2 on the basis of the information indicating the combination, and notifies the target user of the state of the brain waves by changing the resolution of the video, which is the content, on the basis of the state of the brain waves of the target user.

Further, the notification method information indicating the scent notification as the third notification method includes information indicating a combination of “scent” as a type of content used for notifying the state of the brain waves and “change in strength of scent” as a mode of changing components of the content. The scent notification is a notification for causing a scent generator (not shown) that is disposed in the vicinity of the user and capable of generating an aromatic substance to generate an aromatic substance of an amount corresponding to the state of the brain waves of the target user. The scent generator generates an aromatic substance of an amount corresponding to the state of the brain waves of the target user on the basis of the information indicating the combination, thereby notifying the target user of the state of the brain waves. The scent generator has a function of communicating with at least one of the information processing apparatus 1 or the user terminal 2, and the scent generator generates an aromatic substance on the basis of an instruction from the information processing apparatus 1 or the user terminal 2. Here, it is assumed that the aromatic substance output by the scent generator is an aromatic substance corresponding to a scent that is proven to act on the user's brain, such as by awakening or relaxing the user.

The control part 13 is a Central Processing Unit (CPU), for example. The control part 13 functions as the selection part 131, the acquisition part 132, the state identification part 133, and the notification part 134 by executing a program stored in the storage 12.

The selection part 131 selects, from the plurality of user states, an improvement target state that is a user state that is to be improved by the target user whose brain waves are detected. An application program for increasing brain function has been installed in the user terminal 2, for example. In response to the execution of the application program, the user terminal 2 transmits a training start request requesting the start of training brain function to the information processing apparatus 1. Upon receiving the training start request, the selection part 131 causes the user terminal 2 to display a state selection screen for receiving a selection of one of the plurality of user states. The selection part 131 selects the improvement target state by receiving a selection of the improvement target state from the target user via the state selection screen.

Further, the selection part 131 receives, from the target user, a selection of content corresponding to a type of content included in the notification method information associated with the improvement target state selected by the selection part 131. Upon receiving the selection of the improvement target state through the state selection screen, the selection part 131 identifies the analysis method information and the notification method information associated with the improvement target state in the combination information stored in the storage 12, for example. Then, the selection part 131 causes the user terminal 2 to display a content selection screen that receives a selection of one of a plurality of pieces of content corresponding to the type of the content included in the identified notification method information and stored in advance in the storage 12, from the target user. The selection part 131 selects the content by receiving the selection of the content from the target user via the content selection screen. Here, the content that can be selected by the target user may be content stored in the user terminal 2.

It should be noted that the selection part 131 selects the content by receiving the selection of the content from the target user, but it is not limited to this. The selection part 131 may randomly select one of a plurality of pieces of content stored in advance in the storage 12 without receiving the selection of the content from the target user.

The acquisition part 132 acquires brain wave information indicating brain waves of the target user. When the selection part 131 selects the improvement target state and the content, the acquisition part 132 transmits a request to acquire brain wave information to the brain wave measuring device 3 via the user terminal 2, for example. The brain wave measuring device 3 measures the brain waves of the target user in response to receiving the request to acquire the brain wave information. The brain wave measuring device 3 transmits brain wave information indicating the measured brain waves to the information processing apparatus 1 via the user terminal 2. The information processing apparatus 1 acquires the brain wave information by receiving the brain wave information transmitted from the brain wave measuring device 3.

The state identification part 133 identifies the state of the brain waves of the target user indicated by the brain wave information acquired by the acquisition part 132 using the analysis method indicated by the analysis method information stored in the storage 12 in association with the improvement target state selected by the selection part 131. The state identification part 133 analyzes the brain waves of the target user indicated by the brain wave information acquired by the acquisition part 132 using the analysis method indicated by the analysis method information associated with the improvement target state, for example. The state identification part 133 calculates a score indicating the state of the brain waves on the basis of the result of the analysis by this analysis method. The state identification part 133 calculates a score such that the score is higher as the state of the brain waves is better compared to a reference state corresponding to the improvement target state. The state identification part 133 may calculate the score such that the score is higher as the level of improvement is higher than the state of the brain waves of the target user identified in the past.

For example, when the improvement target state selected by the selection part 131 is short-term memory, the state identification part 133 identifies the state of the brain waves of the target user indicated by the brain wave information every predetermined time (for example, one second) by a frequency analysis, which is an analysis method associated with short-term memory, as shown in FIG. 3. The state identification part 133 calculates a score indicating the state of the brain waves on the basis of the intensity of alpha waves included in the brain waves of the target user indicated by the brain wave information identified by the frequency analysis, for example.

The notification part 134 notifies the target user of the state of the brain waves identified by the state identification part 133 on the basis of the notification method indicated by the notification method information stored in the storage 12 in association with the improvement target state selected by the selection part 131. Specifically, the notification part 134 outputs content selected by the selection part 131 to the user terminal 2 over a predetermined training time. The notification part 134 notifies the target user of the state of the brain waves by changing components of the content on the basis of the state of the brain waves identified by the state identification part 133.

More specifically, the notification part 134 identifies a mode of changing the components of the content included in the notification method information stored in association with the selected improvement target state. The notification part 134 notifies the target user of the state of the brain waves identified by the state identification part 133 by changing the components of the content in the identified mode. The notification part 134 notifies the target user of the state of the brain waves identified by the state identification part 133 on the basis of one of the first to fourth notification methods stored in the storage 12 in association with the improvement target selected by the selection part 131.

For example, when the selected improvement target state is short-term memory, the notification part 134 identifies the notification method of the state of the brain waves corresponding to short-term memory as “sound notification and scent notification”, according to the notification method information associated with short-term memory, as shown in FIG. 3. Then, the notification part 134 refers to the information indicating the combination included in the notification method information, and identifies the type of the content as “sound”, and also identifies the mode of changing the components of the content as “change in quality” and “change in strength of scent”. The notification part 134 notifies the target user of the state of the brain waves by performing processing of changing the quality of the sound serving as the content for the user terminal 2 and processing of changing the amount of an aromatic substance generated by the scent generator, on the basis of the state of the brain waves identified by the state identification part 133.

First, the notification part 134 causes the user terminal 2 to output a message indicating a way of training. The notification part 134 instructs the user terminal 2 to notify the target user of a message: “Please be conscious so you can hear the music clearly”, for example.

After that, when the state of the brain waves indicates that the improvement status of short-term memory is good, the notification part 134 instructs the user terminal 2 not to overlap a noise on the sound, and controls the scent generator such that the scent generator generates a scent. Specifically, when the notification part 134 determines that the score indicating the state of the brain waves identified by the state identification part 133 is equal to or greater than a predetermined threshold and the state of the brain waves are good for improving short-term memory, the notification part 134 controls the user terminal 2 not to overlap a noise on the sound, and controls the scent generator such that the scent generator disposed in the vicinity of the user generates a scent. The predetermined threshold is a threshold for determining whether or not the state of the brain waves is good for improving the improvement target state.

On the other hand, when the state of the brain waves indicates that the improvement status of short-term memory is poor, the notification part 134 instructs the user terminal 2 to overlap a noise on the sound and controls the scent generator not to emit an aromatic substance. Specifically, when the notification part 134 determines that the score indicating the state of the brain waves identified by the state identification part 133 is less than a predetermined threshold and the state of the brain waves is poor for improving short-term memory, the notification part 134 controls the user terminal 2 to overlap a noise on the sound and controls the scent generator such that the scent generator does not generate a scent. The notification part 134 may cause the user terminal 2 to overlap a noise having a loudness, a frequency, or a pattern corresponding to a score indicating the state of the brain waves, or may cause the scent generator to generate a scent of an amount corresponding to the score indicating the state of the brain waves or a scent of a type corresponding to the score.

Further, when the selected improvement target state is dementia, the notification part 134 identifies a method for notifying the state of the brain waves with respect to the dementia as “video notification” on the basis of the notification method information associated with dementia, as shown in FIG. 3. Then, the notification part 134 refers to the information indicating the combination included in the notification method information, identifies the type of the content as “video”, and identifies the mode of changing components of the content as “change in quality”.

First, the notification part 134 causes the user terminal 2 to output a message indicating a way of training. The notification part 134 instructs the user terminal 2 to notify the target user of a message: “Please be conscious so you can see the video clearly”, for example.

After that, when the brain waves indicate that the improvement status of dementia is good, the notification part 134 instructs the user terminal 2 not to lower the resolution of the video serving as the content. Specifically, when the notification part 134 determines that the score indicating the state of the brain waves identified by the state identification part 133 is equal to or greater than a predetermined threshold and the state of the brain waves is good for improving dementia, the notification part 134 controls the user terminal 2 to output the video without lowering the resolution of the video.

On the other hand, when the brain waves indicate that the improvement status of dementia is poor, the notification part 134 instructs the user terminal 2 to lower the resolution of the video. Specifically, when the notification part 134 determines that the score indicating the state of the brain waves identified by the state identification part 133 is less than a predetermined threshold and the state of the brain waves is poor for improving dementia, the notification part 134 controls the user terminal 2 to lower the resolution of the video. In this manner, the information processing apparatus 1 can make the target user recognize whether or not the state of the brain waves is good for improving the improvement target state.

It may be desirable to use a different notification method for the improvement target depending on the attributes of the user. When an attribute of the user is “elderly”, the user may have trouble recognizing the state of the brain waves by only the video or the sound, and when an attribute of the user is not “elderly”, it is considered that the user can recognize the state of the brain waves by only the video or the sound, for example.

In contrast, the storage 12 may store the analysis method information, the attribute information indicating the attributes of a plurality of users, and a plurality of pieces of notification method information corresponding to each attribute of the plurality of users, in association with each of a plurality of user states. Further, the information processing apparatus 1 may receive attribute information indicating an attribute of the user from the user of the user terminal 2 in advance, and may store a user ID, given to the user, for identifying the user in association with the attribute information in the storage 12. Here, the attribute of the user is not limited to age information used for identifying whether or not the user is “elderly”, and may include attributes such as sex, occupation, and personality recognized by the user.

In this case, when the selection of the improvement target state is received from the target user, the selection part 131 acquires a user ID of the target user from the user terminal 2, for example. Then, the notification part 134 identifies the attribute of the target user by identifying the attribute information associated with the user ID of the target user acquired by the selection part 131. The notification part 134 notifies the target user of the state of the brain waves identified by the state identification part 133 on the basis of the notification method stored in the storage 12 in association with the improvement target state selected by the selection part 131 and the identified attribute of the target user. In this manner, the information processing apparatus 1 notifies the state of the brain waves in accordance with the attribute of the user, and can effectively perform training of the brain.

Further, a measurable range of states of brain waves is different for each of a plurality of target users. Even if each of the plurality of target users performs the same training, the intensity of brain waves measured may be different, or a range of the brain being activated may be different among the target users, for example.

Accordingly, before notifying the target user of the state of the brain waves, the notification part 134 identifies a range in which the state of the brain waves of the target user changes, on the basis of the state of the brain waves identified by the state identification part 133 in a predetermined time. The predetermined time is a time during which a change in the state of the brain waves can be identified, and is shorter than the training time. Then, the notification part 134 sets a threshold for determining whether the state of the brain waves is good for improving the improvement target state or poor for improving the improvement target state, within the range in which the state of the brain waves changes.

For example, the notification part 134 identifies the maximum value and the minimum value of the score indicating the state of the brain waves identified by the state identification part 133 over a predetermined time, thereby identifying a range in which the state of the brain waves of the target user changes. Then, the notification part 134 sets a predetermined threshold for determining whether or not the state of the brain waves is good for improvement, between the identified maximum value and minimum value.

Then, the notification part 134 identifies a mode corresponding to the result of comparing the state of the brain waves identified by the state identification part 133 to the threshold. The notification part 134 notifies the target user of the state of the brain waves in the identified mode. Specifically, when the notification part 134 determines that the score indicating the state of the brain waves identified by the state identification part 133 is less than a set predetermined threshold and the state of the brain waves is poor for improving the improvement target state, the notification part 134 notifies the user terminal 2 of an instruction to lower the resolution of the video or to overlap a noise on the sound. On the other hand, when the notification part 134 determines that the score indicating the state of the brain waves identified by the state identification part 133 is equal to or greater than the set predetermined threshold and the state of the brain waves is good for improving the improvement target state, the notification part 134 notifies the user terminal 2 of an instruction not to lower the resolution of the video or not to overlap a noise on the sound. In this manner, the information processing apparatus 1 can notify the target user of the state of the brain waves so that the target user can recognize whether or not the state of the brain waves is good, in accordance with the measurement status of the brain waves of the target user.

### [Flow of Processing in Information Processing Apparatus 1]

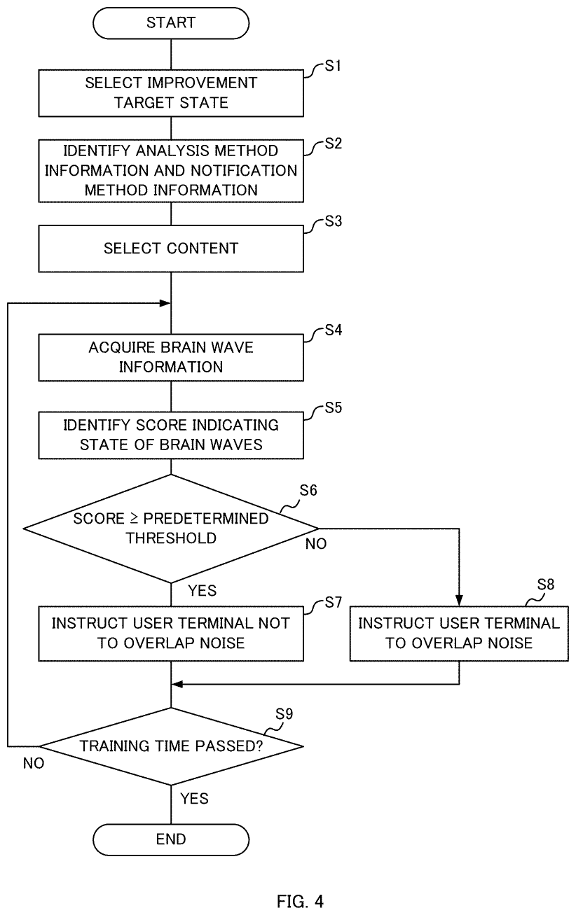

Next, a flow of processing in the information processing apparatus 1 will be described. FIG. 4 is a flowchart showing the flow of processing in the information processing apparatus 1 according to the first embodiment. In this flowchart, it is assumed that the content is a sound, and the state of the brain waves is notified to the target user by whether or not a noise is overlapped on the sound, in order to simplify the description.

First, the selection part 131 selects an improvement target state by receiving a selection of an improvement target state that is a user state that is to be improved by the target user among a plurality of user states, from the target user via the user terminal 2 (S1). Next, the selection part 131 identifies analysis method information and notification method information associated with the selected improvement target state in combination information stored in the storage 12 (S2).

Next, the selection part 131 identifies the type of the content included in the identified notification method information, and receives a selection of the content corresponding to the identified type of the content from the target user, thereby selecting the content to be used for training corresponding to the improvement target state (S3).

Next, the acquisition part 132 acquires brain wave information indicating brain waves of the target user from the brain wave measuring device 3 via the user terminal 2 (S4).

Next, the state identification part 133 identifies a score indicating the state of the brain waves of the target user indicated by the acquired brain wave information through the analysis method indicated by the analysis method information identified in S2 (S5).

Next the notification part 134 determines whether or not the score indicating the state of the brain waves identified in S5 is equal to or greater than a predetermined threshold (S6). When it is determined that the score indicating the state of the brain waves is equal to or greater than the predetermined threshold, the notification part 134 proceeds to S7, and instructs the user terminal 2 not to overlap a noise on the sound serving as the content. When it is determined that the score indicating the state of the brain waves is less than the predetermined threshold, the notification part 134 proceeds to S8, and instructs the user terminal 2 to overlap a noise on the sound serving as the content. As described above, only when the score indicating the state of the brain waves is good, no noise is overlapped on the sound serving as the content, causing the sound to be heard clearly, and thus the target user can recognize that the state of the brain waves is good.

It should be noted that the notification part 134 may change the mode of the noise to be overlapped on the sound in accordance with the score indicating the state of the brain waves. The notification part 134 increases the noise as the score becomes lower, for example. Since the notification part 134 operates in this manner, the target user attempts to change his or her consciousness so as to reduce the noise. Thus, the user is more likely to learn the way of being conscious in such a manner that the state of the brain waves is good, allowing the improvement target state to be improved easily.

Subsequently, the notification part 134 determines whether or not a predetermined training time has passed since the acquisition of the brain wave information started (S9). When it is determined that the training time has passed, the notification part 134 ends the processing according to this flowchart, and when it is determined that the training time has not passed, the notification part 134 proceeds to S4.

### Effects of First Embodiment

As described above, the information processing apparatus 1 according to the first embodiment selects, from the plurality of user states indicating a symptom or ability of a user, an improvement target state that is a user state that is to be improved by a target user whose brain waves are detected, and acquires brain wave information indicating brain waves of the target user. The information processing apparatus 1 identifies the state of the brain waves of the target user indicated by the brain wave information according to an analysis method indicated by analysis method information stored in the storage 12 in association with the selected improvement target state, and notifies the target user of the identified state of the brain waves on the basis of the notification method indicated by notification method information stored in the storage 12 in association with the selected improvement target state. In this manner, the information processing apparatus 1 can perform training for increasing brain function in accordance with an ability or symptom the user wants to improve.

### Second Embodiment

### [Identifying Content Suitable for Training Brain Waves]

Next, the second embodiment will be described. In the information processing apparatus 1 according to the first embodiment, it is assumed that the selection part 131 selects content by receiving a selection of content from a target user or randomly selects content. On the other hand, the second embodiment is different from the first embodiment in that content suitable for training for increasing brain function is identified and used for the training. The information processing apparatus 1 according to the second embodiment will be described below. The description of the same parts as those of the first embodiment will be omitted as appropriate.

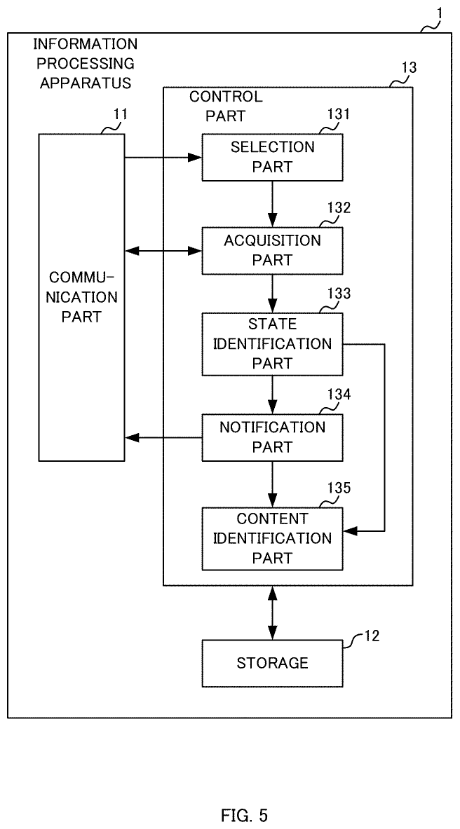

FIG. 5 shows a configuration of the information processing apparatus 1 according to the second embodiment. As shown in FIG. 5, the control part 13 of the information processing apparatus 1 according to the second embodiment includes a content identification part 135.

Among the plurality of pieces of content stored in the storage 12, the notification part 134 according to the second embodiment outputs any of one or more pieces of content associated with the type of content included in the notification method information associated with the improvement target state selected by the selection part 131 to the user terminal 2. Then, on the basis of the state of the brain waves of the target user identified by the state identification part 133, the notification part 134 changes components of the content in a mode in which the components of the content included in the notification method information are changed, thereby notifying the target user of the state of the brain waves.

After training of brain functions has been performed a plurality of times by the target user and a plurality of pieces of content have been output by the notification part 134, the content identification part 135 identifies effective content, which is content suitable for training for increasing brain function. Specifically, on the basis of the state of the brain waves of the target user identified by the state identification part 133 when each of the plurality of pieces of content is output in accordance with the improvement target state, the content identification part 135 identifies, as effective content, content with a relatively high frequency of causing the appearance of a state in which the brain waves have a high improvement effect for the improvement target state, among the plurality of pieces of content.

The notification part 134 outputs the identified effective content to the user terminal 2 in response to the identification of the effective content in accordance with the improvement target state by the content identification part 135. Specifically, after the effective content is identified for the improvement target state by the content identification part 135, when the training for increasing the brain function corresponding to the improvement target state is newly performed, the notification part 134 outputs the effective content to the user terminal 2 and performs the training.

It should be noted that the notification part 134 may identify the attribute of the content stored in the storage 12 in association with the effective content identified by the content identification part 135. Then, the notification part 134 may output any of one or more pieces of content stored in the storage 12 in association with the identified attribute to the user terminal 2 to perform training. In this manner, the information processing apparatus 1 according to the second embodiment outputs effective content and performs training, thereby increasing the appearance frequency of a state of brain waves having a high improvement effect for the improvement target state, and making it possible to efficiently increase the brain function.

### Third Embodiment

### [Identifying Analysis Method Suitable for Improvement Target]

Next, the third embodiment will be described. The information processing apparatus 1 according to the third embodiment differs from the first embodiment in that an analysis method suitable for an improvement target is identified.

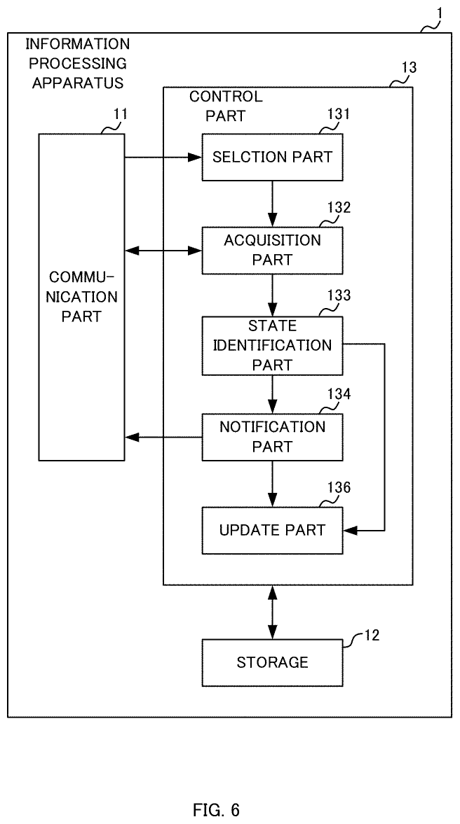

FIG. 6 shows a configuration of the information processing apparatus 1 according to the third embodiment. As shown in FIG. 6, the control part 13 of the information processing apparatus 1 according to the third embodiment includes an update part 136.

Before training of brain function is performed for a plurality of target users, the storage 12 according to the third embodiment stores a plurality of pieces of analysis method information in each of the plurality of user states. Due to this, an analysis of the brain waves is performed by one of a plurality of analysis methods with respect to one improvement target state. That is, the state identification part 133 identifies the state of the brain waves of the target user indicated by the brain wave information, through the analysis method indicated by one of the plurality of pieces of the analysis method information stored in the storage 12 in association with the improvement target state selected by the selection part 131. After that, the notification part 134 notifies the target user of the state of the brain waves identified by the state identification part 133 on the basis of the notification method indicated by the notification method information stored in the storage 12 in association with the improvement target state selected by the selection part 131.

The update part 136 acquires the first status information and the second status information. The first status information is information indicating the status of the improvement target state of the target user before the notification part 134 notifies the state of the brain waves. The second status information is information indicating the status of the improvement target state of the target user after the notification part 134 has notified the state of the brain waves according to the analysis method indicated by one of the plurality of pieces of analysis method information. The update part 136 performs a test for identifying the status of the improvement target state to the target user before and after the notification part 134 notifies the state of the brain waves, respectively, and acquires the first status information and the second status information, for example. The first status information and the second status information may be the score of the test, for example.

The update part 136 acquires a plurality of pieces of second status information according to each of the plurality of pieces of analysis method information when a plurality of pieces of analysis method information are selected. Then, on the basis of the acquired first status information and the plurality of pieces of the second status information, the update part 136 identifies the second status information indicating that the degree of improvement of the improvement target state is relatively high, among the plurality of pieces of the second status information. The update part 136 identifies the second status information with a score in which the difference from the score of the test indicated by the first status information is relatively large, as the second status information indicating that the degree of improvement of the improvement target state is relatively high, for example. The update part 136 identifies the analysis method used when the identified second status information is acquired as an analysis method suitable for improving the improvement target state. The update part 136 determines that the analysis method associated with this improvement target state should be only the identified analysis method, and limits the analysis method stored in the storage 12 associated with the improvement target state to the identified analysis method.

In this manner, after identifying the analysis method suitable for improving the improvement target state among the plurality of analysis methods associated with the improvement target, the information processing apparatus 1 according to the third embodiment can analyze the brain waves using this analysis method, making it possible to efficiently improve the improvement target state of the target user.

The present disclosure is explained on the basis of the exemplary embodiments. The technical scope of the present disclosure is not limited to the scope explained in the above embodiments and it is possible to make various changes and modifications within the scope of the disclosure. For example, all or part of the apparatus can be configured to be functionally or physically distributed and integrated in arbitrary units. For example, in the above embodiment, the information processing apparatus 1 connected to the user terminal 2 in a manner enabling communication functions as the selection part 131, the acquisition part 132, the state identification part 133, and the notification part 134, but is not limited to this. For example, a training program functioning as the selection part 131, the acquisition part 132, the state identification part 133, and the notification part 134 may be stored in the user terminal 2, and the user terminal 2 may function as the selection part 131, the acquisition part 132, the state identification part 133, and the notification part 134 by executing the training program. Further, the information processing apparatus 1 and the user terminal 2 may cooperate to function as the selection part 131, the acquisition part 132, the state identification part 133, and the notification part 134.

Further, new exemplary embodiments generated by arbitrary combinations of them are included in the exemplary embodiments. Further, effects of the new exemplary embodiments brought by the combinations also have the effects of the original exemplary embodiments.

What is claimed is:

1. An information processing apparatus comprising:
a storage that stores analysis method information indicating an analysis method corresponding to an analysis of brain waves of a user and notification method information indicating a notification method for notifying the user of a state of the brain waves of the user identified by the analysis, in association with each of a plurality of user states indicating a symptom or ability of the user;
a selection part that selects an improvement target state that is a user state that is to be improved by a target user whose brain waves are detected, among the plurality of user states;
an acquisition part that acquires brain wave information indicating brain waves of the target user;
a state identification part that identifies a state of the brain waves of the target user indicated by the brain wave information according to an analysis method indicated by the analysis method information stored in the storage in association with an improvement target state selected by the selection part; and
a notification part that notifies the target user of the state of the brain waves identified by the state identification part on the basis of the notification method indicated by the notification method information stored in the storage in association with the improvement target state selected by the selection part.

2. The information processing apparatus according to claim 1, wherein
the storage stores the analysis method information, attribute information indicating attributes of a plurality of users, and a plurality of pieces of the notification method information corresponding to each of the attributes of the plurality of users in association with each of the plurality of user states, and stores user identification information for identifying a user in association with attribute information indicating the attribute of the user, and
the notification part identifies an attribute of the target user by identifying the attribute information associated with the user identification information of the target user, and notifies the target user of the state of the brain waves identified by the state identification part on the basis of the notification method stored in the storage in association with the improvement target state selected by the selection part and the identified attribute of the target user.

3. The information processing apparatus according to claim 1, wherein
the notification method information includes information indicating a combination of a type of content used for notifying the state of the brain waves and a mode of changing components of the content on the basis of the state of the brain waves,
the selection part receives, from the target user, a selection of content corresponding to a type of content included in notification method information associated with the improvement target state selected by the selection part, and
the notification part outputs the content selected by the selection part to the user terminal, and notifies the target user of the state of the brain waves identified by the state identification part by changing the components of the content in a mode in which the components of the content indicated by the notification method information stored in the storage in association with the improvement target state are changed.

4. The information processing apparatus according to claim 1, wherein
the notification method information includes information indicating a combination of a type of content used for notifying the state of the brain waves and a mode of changing components of the content on the basis of the state of the brain waves,
the storage stores a plurality of pieces of content and a type of the content in association with each other,
among the plurality of pieces of content stored in the storage, the notification part outputs any of one or more pieces of content associated with a type of content included in the notification method information associated with the improvement target state selected by the selection part to the user terminal, and notifies the target user of the state of the brain waves identified by the state identification part by changing components of the content in a mode in which the components of the content included in the notification method information are changed, and
the information processing apparatus further includes a content identification part that identifies, as effective content, content with a relatively high frequency of causing an appearance of a state of brain waves having a high improvement effect for an improvement target state among the plurality of pieces of content, on the basis of the state of the brain waves of the target user identified by the state identification part when each of the plurality of pieces of content is output, wherein
the notification part outputs the identified effective content to the user terminal in response to identification of the effective content by the content identification part.

5. The information processing apparatus according to claim 4, wherein
the notification part identifies an attribute stored in the storage in association with the effective content, and outputs one or more pieces of content stored in the storage in association with the identified attribute to the user terminal.

6. The information processing apparatus according to claim 1, wherein
the storage stores a plurality of pieces of the analysis method information in association with each of the plurality of user states,
the state identification part identifies the state of the brain waves of the target user indicated by the brain wave information according to an analysis method indicated by one of a plurality of pieces of the analysis method information stored in the storage in association with the improvement target state selected by the selection part, and
the information processing apparatus further includes an update part that acquires first status information indicating status of the improvement target state of the target user before the notification part notifies the state of the brain waves, and second status information indicating status of the improvement target state of the target user after the notification part notifies the state of the brain waves obtained by analyzing the state of the brain waves using an analysis method indicated by one of a plurality of pieces of the analysis method information, and identifies the second status information indicating that the degree of improvement of the improvement target state is relatively high on the basis of the first status information and the second status information, and identifies an analysis method used when the identified second status information is acquired as an analysis method suitable for improving the improvement target state, and limits the analysis method stored in the storage associated with the improvement target state to the identified analysis method.

7. The information processing apparatus according to claim 1, wherein
before notifying the target user of the state of the brain waves, the notification part identifies a range in which the state of the brain waves of the target user changes, on the basis of the state of the brain waves identified by the state identification part in a predetermined time, and sets a threshold for determining whether the state of the brain waves is good for improving the improvement target state or the state of the brain waves is poor for improving the improvement target state, in the range in which the state changes, and notifies the target user of the state of the brain waves in a mode corresponding to a result of comparing the state of the brain waves identified by the state identification part to the threshold.

8. The information processing apparatus according to claim 1, wherein
a plurality of notification methods includes:
a first notification method for notifying the state of the brain waves by outputting a sound, which is content, to the user terminal and adding a noise to the content on the basis of the state of the brain waves of the target user;
a second notification method for outputting a video, which is content, to the user terminal and changing resolution of the content on the basis of the state of the brain waves of the target user;
a third notification method for causing a device capable of generating an aromatic substance disposed in the vicinity of the user to generate an aromatic substance on the basis of the state of the brain waves of the target user; and
a fourth notification method obtained by combining at least two notification methods from the first notification method to the third notification method,
wherein
the notification part notifies the target user of the state of the brain waves identified by the state identification part on the basis of one of the notification methods from the first notification method to the fourth notification method stored in the storage in association with the improvement target selected by the selection part.

9. The information processing apparatus according to claim 8, wherein
in a case where the state of the brain waves is notified to the target user on the basis of the first notification method, when the state of the brain waves indicates a poor state, the notification part adds more noises to be added to the content compared to when the state of the brain waves indicates a good state.

10. The information processing apparatus according to claim 8, wherein
the notification part calculates a score such that the score becomes higher as the state of the brain waves is better, and increases the noise as the calculated score is lower.

11. The information processing apparatus according to claim 8, wherein
in a case where the state of the brain waves is notified to the target user on the basis of the second notification method, the notification part lowers the resolution of the content when the state of the brain waves indicates a poor state compared to when the state of the brain waves indicates a good state.

12. The information processing apparatus according to claim 11, wherein
the notification part does not lower the resolution of the content when the state of the brain waves is good.

13. The information processing apparatus according to claim 8, wherein
the aromatic substance is an aromatic substance that awakens or relaxes the user, and
in a case where the state of the brain waves is notified to the target user on the basis of the third notification method, the notification part reduces an amount of an aromatic substance generated by the device capable of generating the aromatic substance when the state of the brain waves indicates a poor state, compared to when the state of the brain waves indicates a good state.

14. The information processing apparatus according to claim 13, wherein
the notification part causes the aromatic substance to be generated when the state of the brain waves is good, and does not cause the aromatic substance to be generated when the state of the brain waves indicates a poor state.

15. An information processing method executed by a computer, comprising the steps of:
selecting an improvement target state that is a user state that is to be improved by a target user whose brain waves are detected, from a plurality of user states indicating a symptom or ability of a user;
acquiring brain wave information indicating brain waves of the target user;
identifying a state of the brain waves of the target user indicated by the brain wave information according to an analysis method indicated by the analysis method information stored in association with the selected improvement target state, in a storage that stores analysis method information indicating an analysis method corresponding to an analysis of brain waves of the user and notification method information indicating a notification method for notifying the user of a state of brain waves of the user identified by the analysis, in association with each of a plurality of user states; and
notifying the target user of the identified state of the brain waves on the basis of the notification method indicated by the notification method information stored in the storage in association with the selected improvement target state.

16. A non-transitory computer-readable storage medium storing a program for causing a computer to function as:
a selection part that selects an improvement target state that is a user state that is to be improved by a target user whose brain waves are detected, from a plurality of user states indicating a symptom or ability of a user;
an acquisition part that acquires brain wave information indicating brain waves of the target user;
a state identification part that identifies a state of the brain waves of the target user indicated by the brain wave information according to an analysis method indicated by the analysis method information stored in association with the selected improvement target state, in a storage that stores analysis method information indicating an analysis method corresponding to an analysis of the brain waves of the user and notification method information indicating a notification method for notifying the user of a state of the brain waves of the user identified by the analysis in association with each of the plurality of user states; and
a notification part that notifies the target user of the identified state of the brain waves on the basis of the notification method indicated by the notification method information stored in the storage in association with the selected improvement target state.
